(19) United States
(12) Patent Application Publication
(10) Pub. No.: US 2010/0069252 A1
(43) Pub. Date: Mar. 18, 2010

(54) EFFICIENT METHOD FOR PARTIAL SEQUENCING OF PEPTIDE/PROTEIN USING ACID OR BASE LABILE XANTHATES

(76) Inventor: Bakshy Akshaykirit Chibber, Mishawaka, IN (US)

Correspondence Address: ICE MILLER LLP, ONE AMERICAN SQUARE, SUITE 3100, INDIANAPOLIS, IN, 46282-0200, US

(21) Appl. No.: 12/513,031

(22) PCT Filed: Oct. 30, 2007
(86) PCT No.: PCT/US07/83032

§ 371 Date: Apr. 30, 2009

Related U.S. Application Data
(60) Provisional application No. 60/863,570, filed on Oct. 30, 2006.

(51) Int. Cl. G01N 33/68; C40B 60/10; C40B 30/00

(52) U.S. Cl. 506/7; 506/38; 436/86

(57) ABSTRACT

A method and system for sequencing polypeptides utilizing acid and base labile xanthates.

### CLAIM OF PRIORITY

This application is a nationalization of International Application PCT/US07/83032, published as WO 2008/073599 and having a priority date of 30 Oct. 2006, as the International Application claims priority to U.S. Provisional Patent Application No. 60/863,570 and titled EFFICIENT METHOD FOR PARTIAL SEQUENCING OF PEPTIDE/PROTEIN USING ACID OR BASE XANTHATES, filed Oct. 30, 2006.

### BACKGROUND

The present application relates to methods for sequencing of peptides and/or proteins, by using acid and/or base labile xanthates and more particularly application of these labile xanthates for the generation of protein and peptide ladders concomitant with the sequential chemical removal of free amino-acids from the N-terminal of proteins or peptides.

Protein sequencing is used in many biochemical, pharmaceutical, and biomedical research fields to determine partial or whole amino acid composition of a sample protein, in addition to the sequence in which those amino acids take within a given protein. By determining the amino acid sequence of a new protein, its structural and biological function can be better known. Further, an unknown sample protein can be readily identified as a previously known protein through the use of protein sequencing.

Protein sequencing can be performed in a number of different manners, from the use of the Edman degradation reaction, thioacylation, or the use of mass spectrometry, matrix assisted laser desorption ionization, or electrospray ionization (ESI). A brief description of each of these sequencing methods follows, with a:

I. The Edman Degradation Process

The Edman degradation process, first described by P. Edman, is the basis for modern chemical peptide sequencing. The Edman degradation process operates by removing and identifying each amino acid from the N-terminal end of a protein, thereby allowing a practitioner to identify the composition and sequence of a particular protein. (P. Edman, ACTA. CHEM SCAND. 10,761 (1957)). More specifically, three reactions are used in the Edman degradation process to remove each N-terminal amino acid: (1) coupling, (2) cleavage, and (3) conversion.

The first reaction, often referred to as coupling, modifies the N-terminal amino acid by adding phenylisothiocyanate (“PITC”) to the amino group, typically in a base-catalyzed reaction. The result of coupling is a phenylthiocarbamoyl (“PTC”) protein with the PTC-coupled amino acid occurring at the N-terminal end of the protein. This PTC-coupled amino acid can then be subjected to the second reaction, cleavage, to remove the PTC-coupled amino acid from the protein. The cleavage reaction is typically performed by treating the PTC protein with an anhydrous acid, thereby allowing the sulfur from the PTC group to react with the first carbonyl carbon in the protein chain. As such, this cyclization reaction results in the removal of the first amino acid as an 2-anilino-5(4)-thiozolinone (“ATZ”) derivative, thereby exposing the next N-terminal amino acid on the protein. At this point the, cleaved amino acid, as an ATZ derivative, can be extracted from the residual polypeptide. The cleaved amino acid is then subjected to the third reaction, conversion, wherein the ATZ derivative is converted to a phenylthiohydantoin (“PTH”) amino acid (the “converted amino acid”) by exposing the ATZ derivative to heat and an aqueous or other protic acid environment. The PTH amino acid is more stable and allows for analyzing and identification of the amino acid.

Identification of the PTH amino acid derivative may be performed by either using fluorescent reagents that attach to the cleaved PTH amino acid derivative, or by using fluorescent reagents in the earlier steps of the Edman process to cause a fluorescent-coupled ThioHydantoin amino acid derivative. However, such reactions are slow, and may result in low percentages of fluorescent coupled amino acid derivatives due to the fact that fluorescent reagents tend to have unfavorable electron configuration. As a result, other methods of identification, including the use of gas liquid chromatography such as high pressure liquid chromatography (“HPLC”), surface phase microextraction chromatography, or mass spectrometry may be used to identify the PTH amino acid derivative.

According to the Edman degradation process, the process of coupling, cleaving, and then converting and identifying the amino acid from the remaining polypeptide is then continued in an iterative fashion until each of the amino acids comprising the original protein have been removed from the N-terminal end and identified.

II. Thioacylation Protein Sequencing

As an alternative to the Edman degradation process, thioacylation allows the use of relatively mild conditions and faster reactions than the original Edman degradation. Typical thioacylation sequencing involves three steps, similar to the Edman degradation, but the coupling step results in attaching the N-terminal amino acid to an insoluble support, allowing for solid phase chemistry to be utilized.

A more complete discussion of thioacylation degradation can be found in U.S. Pat. No. 5,246,865 to Stolowitz et al. (the “Stolowitz Patent”), which is incorporated by reference herein. The Stolowitz Patent indicates that the most of the proposed compounds used for thioacylation have a lower reactivity than the PITC utilized in the Edman degradation process. The Stolowitz Patent discloses the use of more generally available reagents that display better reactivity than the previous thioacylating compounds and allow for better deposition of the cleaved amino acid complexes on a hydrophobic membrane. Thus, the method disclosed in the Stolowitz Patent allows for a more sensitive sequencing system due to the increased reactivity and better retention on a hydrophobic film layer. Further, gas chromatography, mass spectrometry, or chemical ionization mass spectrometry can be used to identify each amino acid complex that is removed from the polypeptide or protein in each iteration of the degradation reaction by the thioacylation protein sequencing process. However, the method disclosed in the Stolowitz Patent utilizes reactants that may modify the side chains of amino acids, making proper sequencing difficult.

III. Mass Spectrometry

Protein sequence identification through the use of mass spectrometry alone is used in many chemical identification applications by measuring the ratio between the mass and charge of a sample. Protein fragmentation and substantial bioinformatics computing power is required to perform such an analysis. Further, mass spectrometry protein sequencing cannot accurately identify large proteins without modification of the proteins, either through ionization of the proteins (usually performed through electrospray ionization), or chemical or enzymatic digestion of the proteins into smaller polypeptides, each of which may cause the transformation of certain amino acids.

Variations of protein sequencing using mass spectrometry include ladder sequencing. Ladder sequencing utilizes mass spectrometry to compare the resultant peptides that are given off after sequential digesting of proteins. The digestion process may be performed using enzymatic techniques that cleave a protein into multiple polypeptides, or as a modified Edman chemical degradation.

Several methods for performing ladder sequencing may be used, including the use of exopeptidases to cleave off terminal amino acids or dipeptides. This technique has limited application due to the variability of reactivity with respect to the target protein. Alternatively, PITC with a low percentage of phenylisocyanate (“PIC”) has been used to generate several peptide fragments that can be compared to statistically determine the sequence of the protein in a mixture. This PITC/PIC method has the disadvantage of resulting in a substantial loss of peptides during washing cycles, and reducing the effectiveness of ionization of the products, which can significantly alter the effectiveness of sequencing when small protein sample sizes are utilized.

As will be appreciated, the multiple approaches taken to protein sequencing have been made in an attempt to produce a protein sequencing system that: can be used with high sensitivity so that small samples can be accurately sequenced, can be used on a broad range of proteins without selectivity issues; and which allows a higher throughput of samples to allow protein sequencing to be used on a larger and more efficient scale. However, the several approaches noted above do not allow large sample sizes to be run in short time periods due to the multiple iterations of cycling required under the Edman process and its related methods, and due to the focus on obtaining high sensitivity in sequencing results. Conversely a reliable, high throughput system would greatly appreciate state of the art in protein sequencing to allow rapid qualitative identification of protein or peptide via their sequences.

IV. Automated Sequencing

As will be appreciated from the above discussion of protein sequencing, the processes involved in any degradation or enzymatic digestion sequencing is repetitive and can be time consuming—particularly when small sample sizes are involved and care must be taken not to lose a substantial amount of the sample during processing. As such, automated chemical systems have been developed to perform such tasks.

V. Perspectives on Sequencing Types

As discussed above, the Edman degradation reactions are an established method for the sequential degradation of proteins. Three reactions are required to remove the amino-terminal amino acid and convert it to a form which is suitable for analysis. The first reaction (coupling) modifies the amino terminus by the addition of phenylisothiocyanate (PITC) to the amino group. This is usually a base-catalyzed reaction. The resulting phenylthiocarbamyl (PTC) protein is then treated with an anhydrous acid in a second reaction (cleavage) which allows the sulfur from the PTC group to react with the first carbonyl carbon in the protein chain. This cyclization reaction results in the removal of the first amino acid as an anilinothiozolinone (ATZ) derivative and leaves the next amino acid in the protein exposed for the next round of PITC coupling. In a third reaction (conversion), the ATZ amino acid is converted to a phenylthiohydantoin (PTH) amino acid in aqueous acid. The PTH is more stable than the ATZ and can be easily analyzed. The PTH is a relatively stable form of the amino acid and is readily generated from the products of the Edman acid cleavage step by treatment with aqueous acid. This conversion reaction provides an easy way to obtain a single PTH amino acid derivative from the mixture of ATZ, PTC and PTH amino acids which are present after the cleavage step. Further, the detection of PTH amino acid derivative is done by using fluorescent Edman reagents but they suffer from drawbacks such as either slow coupling or slow cleavage reactions due to the unfavorable electronic configuration or the bulky nature of the fluorescent group.

The thioacylation degradation of proteins and polypeptides was first proposed by Barrett (Barrett, G. C. (1967) Chem. Comm. 487) as an alternative to the Edman degradation. The process involves reacting the N-terminal amino acid of a starting polypeptide immobilized on an insoluble support by adsorption or covalent attachment with a thioacylating reagent.

Thioacylation offers some advantages over the Edman degradation in that the cleavage reaction is short in duration and occurs under relatively mild conditions. Also liberated during the cleavage reaction is the salt of the residual polypeptide, which is the starting polypeptide with the N-terminal amino acid removed. Various reagents including S-(carboxymethyl) dithiobenzoate (CMBTB), S-(cyanomethyl dithiobenzoate, m-nitrobenzoylthionocholine and N-thiobenzoylsuccinimide have been proposed for the sequential degradation of polypeptides by the thioacylation method. Several of aforementioned compounds are not as reactive as PITC and this constituted an important drawback for the development of a satisfactory procedure for sequential analysis.

Further, another method of sequencing proteins and peptides by thiobenzoylation of the protein or peptide, followed by cleavage, and then conversion to a detectable and stable species is disclosed in U.S. Pat. No. 5,246,865. The method and chemistry therein, however, causes modification of the side chains of the amino acids during sequencing.

Ladder sequencing, in which a sequence is read by the mass difference between sequential degradation products, was developed first as an enzymatic technique and then subsequently as a modified Edman type chemical degradation. The use of exopepetidases to generate ladders is limited by the extreme variability of the activity of the protease toward the substrate and is only useful for individual isolated peptides. Ladder chemical sequencing may be performed using phenylisothiocyanate with a small percentage of phenylisocyanate as a chain-terminating regent. The main disadvantages of the methods are the loss of peptide during the washing steps, which limit the sensitivity, as well as the terminating reagent, which removes the alpha N-terminus as a charge carrier, thereby diminishing the relative effectiveness of ionization. The volatile trifluoroethylisothiocyanante analogue removes the need for washes with organic solvent but require that the parent peptide be added back in aliquots in order to generate the ladder which again causes losses through sample handling.

### SUMMARY OF THE INVENTION

According to at least one embodiment, the primary structural analysis of proteins proceeds in two steps, starting with sequence analysis of the N-terminus and proceeding to the sequence analysis of internal peptides generated by proteolytic or enzymatic cleavage of protein into fragments. In one exemplary embodiment, methods of protein sequence determination evolved at a time when data on gene sequences was almost non existent.

In yet another embodiment, an extensive sequence database of known gene and protein sequences,(e.g., the human genome) is utilized to reduce the number of residues that must be chemically sequenced to identify a protein. Further, according to at least one aspect of the present application it is recognized that sequencing genes utilizing limited protein sequence information is a more rapid and desirable means of obtaining complete protein sequence information, and thus allows less chemically efficient agents for sequential protein sequencing to become more desirable for the purpose of obtaining rapid and limited protein sequence information.

On the contrary, in Edman sequencing chemistry, amino acids from the amino terminus are sequentially cleaved from the peptide chain as their anilinothiozolinone derivatives; however, these derivatives are unstable and undergo a number of reactions. Accordingly, according to at least one embodiment, a method for partial sequencing of proteins and peptides using acid and base labile xanthates which also provide free N-terminal amino-acids with ease are surprisingly found desirable for making ladder and subtractive methods utilizing mass spectroscopy.

### DETAILED DESCRIPTION

According to at least one embodiment, reactive O-Tertiary Butyl Xanthatoylating and O-(9-H-Fluoren-9-yl) Xanthoylating reagents are utilized for N-terminal chemical sequencing. These reagents, upon reacting with free α amino group of amino-acids, peptides and proteins provide, respectively, form O-tertiary butyl and O-(9-H-Fluoren-9-yl) Xanthamido derivatives, respectively. According to at least one embodiment, derivatives include sulfur analogs of the common acid and base labile N-tBOC and N-FMOC groups, respectively, which are extensively employed in amino acid, peptide, protein and organic chemistry as N-protecting groups, wherein the carbonyl function is replaced by the thiocarbonyl function of the xanthates. The process whereby these acid and base labile Xanthates are prepared and used in protein and peptide N terminal sequencing is illustrated below:

### Synthesis of O-t Butyl Xanthate Thio Esters for N-Terminal Sequencing

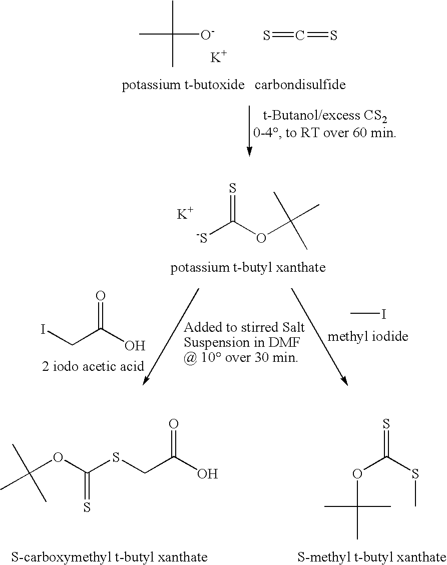

According to at least one embodiment, the above reactions are used to obtain reactive O-alkyl and O-aryl xanthate esters for N-terminal sequencing of proteins and peptides. The S-methyl esters can be converted to xanthoyl imidazoles by treatment with imadazole. O-phenyl methyl xanthate is prepared similarly, starting from potassium phenolate. O-phenyl methyl xanthate was selected in this exemplary embodiment because, upon reaction with the N-terminal amino group of peptides and proteins the reaction yields an O-phenylxanthamido derivative isoelectronic with the phenylthiourea obtained in the corresponding reactions utilizing Edman's reagent (phenyl isothiocyanate) in protein and peptide sequencing, as shown below:

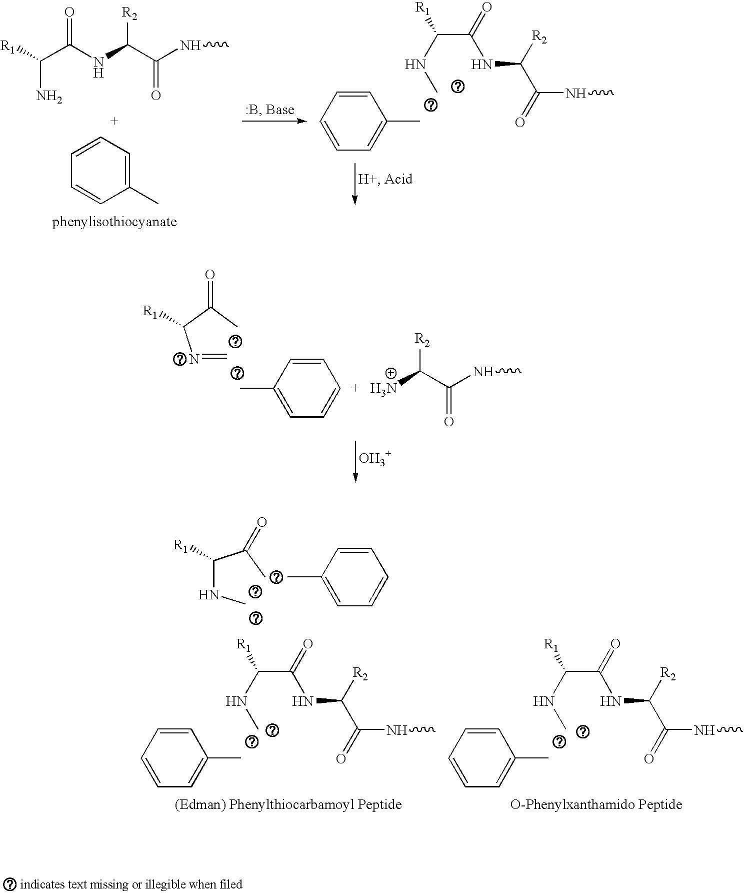

### Protein & Peptide Sequencing Using Acid Labile Methyl O-t Butyl Xanthate

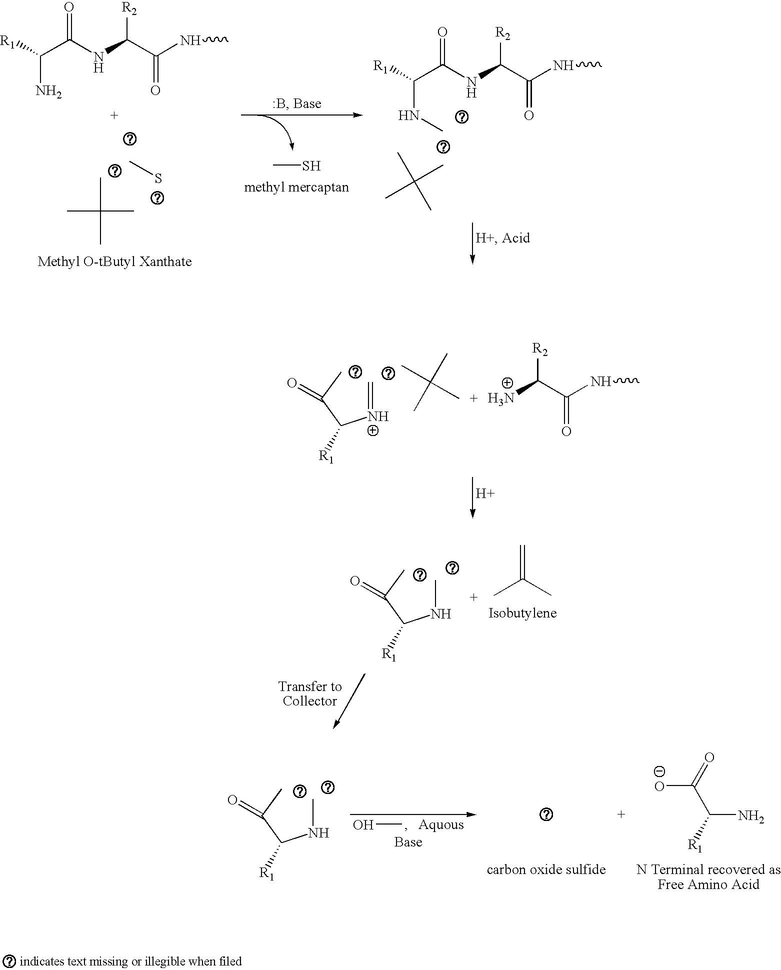

Reactions at Lysine Side Chains during Methyl O-t Butyl Xanthate Sequencing

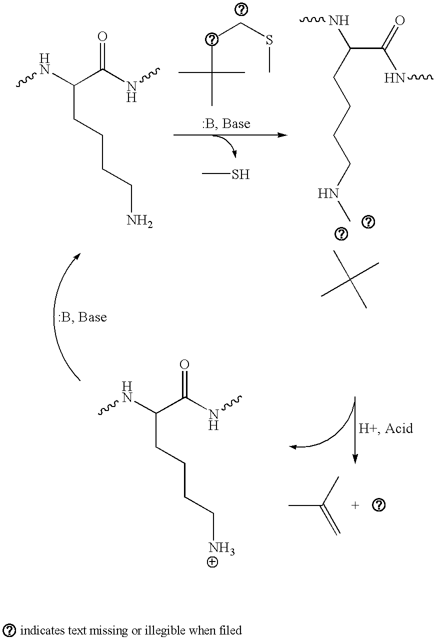

### Protein & Peptide Sequencing Using Base Labile Methyl O-Fluorenylmethyl Xanthate

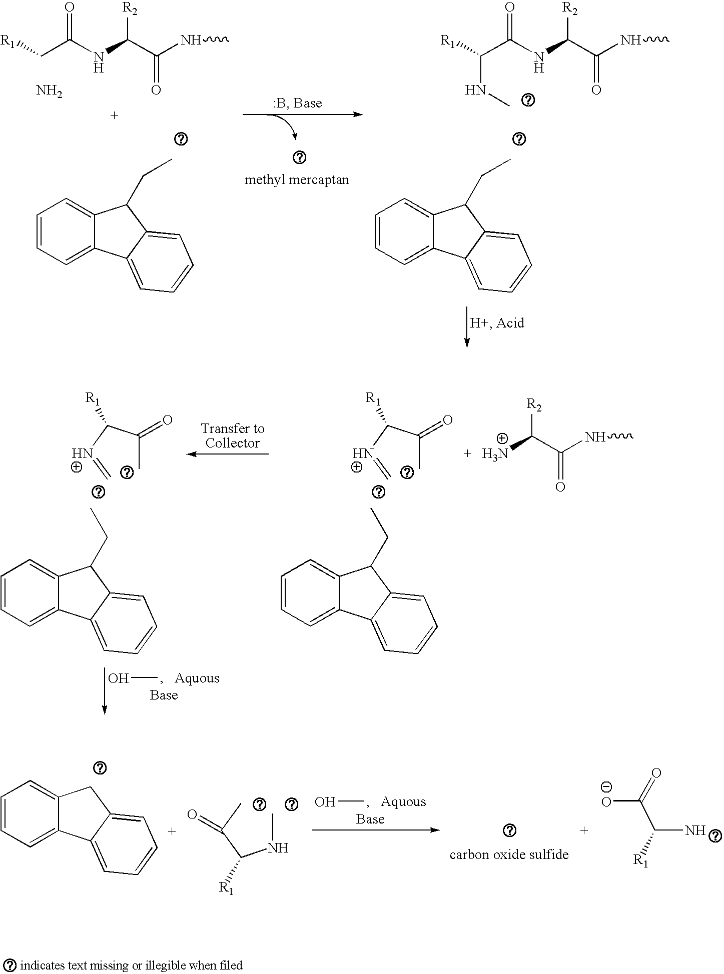

The above reagent, Methyl O-Fluorenylmethyl Xanthate, may conveniently be obtained according to the procedure shown below:

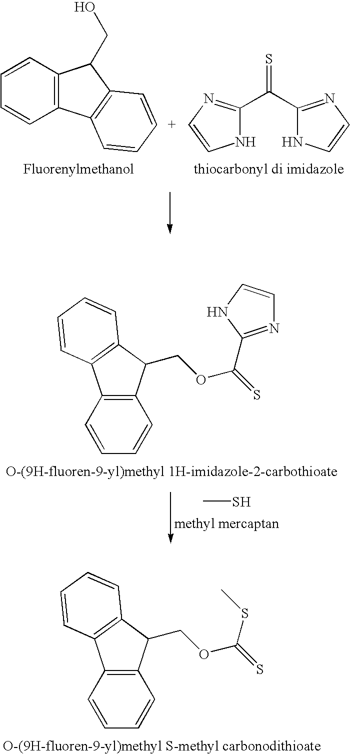

Such activated Xanthates may be synthesized with conventional leaving groups (X), where —X may be ═—Cl,

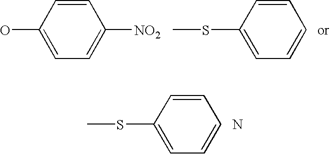

It will be appreciated that additional substituents may be substituted to provide additional activated Xanthates.

One embodiment of the present application relates to utilizing a known DNA, RNA, or protein library to act as a known set of sequences against which unknown proteins may be compared for identification. In particular, the identification of proteins or polypeptides from a particular organism or group of organisms (such as populations, subspecies, species, genera, etc.) is used to narrow the universe of potential proteins that are being tested to a discrete protein population. By way of nonlimiting example, a DNA sample, RNA sample, or array of proteins from the organism or groups of organisms may be used to form or extrapolate a library of protein sequences of the relevant protein population for later identification of an unknown protein sample or samples. It will be appreciated that protein population libraries can be identified by using previous methods of DNA or RNA sequencing, mass spectroscopy, or in depth protein sequencing. Because the libraries of genomes for various organisms are now available from many different sources, a protein population for an organism or group of organisms may be readily available, and relevant proteins may be identified without any sequencing performed prior to testing unknown samples.

In one embodiment, a system for identifying proteins comprises the cleavage of at least five or more N-terminal or C-terminal amino acids from an unknown protein using chemistries discussed above in which acid or base labile Xanthates are prepared and used in protein and peptide N terminal sequencing is illustrated above.

For example, five or more coupling and cleavage reactions may be cyclically performed to remove the first five or more amino acids from the unknown protein. After each cycle, the cleaved amino acid may be washed off from the reaction vessel and identified. The identification of the first five or more amino acids is then recorded as a partial sequence, and that partial sequence is compared to the protein sequence library previously discussed.

It will be appreciated that several proteins from the protein population represented in the protein sequence library may have identical partial amino acid sequences of five or more residues to that of the unknown sample. This is one reason why the Edman process and other processes previously used require sequencing by identifying long sequences or each amino acid in sequence in an unknown sample to identify the polypeptide or protein. However, according to one aspect of the present application, the molecular weight of each unknown sample is also taken and compared against the molecular weight of the population of proteins identified by comparing the first five or more amino acids of the unknown sample with the protein sequence library. In this manner, when both the sequence of the first five or more amino acids of an unknown sample and its molecular weight are compared to the sequence of the first five or more amino acids and molecular weights of the protein population accumulated for the protein sequence library, nearly all unknown samples from a particular organism can be identified simply by comparing the discovered sequence and molecular weight. Furthermore, in instances where the starting sequence of five or more amino acids and the molecular weight of the protein fail to match, that provides valuable information relating to post-translational processing and/or modification of the protein under investigation. As such, identification through limited sequencing can be accomplished without exhaustive and iterative sequencing. In the event that the first five or more amino acids and molecular weight cannot positively identify the unknown sample as a single protein or peptide from the identified protein population, additional sequencing may be performed on the unknown sample. In the event that several proteins with identical sequences of the first five amino acids are identified in a protein sequencing library, the first 10 or fewer, or the first 20 or fewer amino acids for each sample could be taken. However, a significant reduction in time taken to identify the samples would be appreciated even if only half of the unknown samples were immediately identifiable through the comparison method.

It will be appreciated that alternative embodiments in which sequence of the first 5 or more amino acids from the N-terminal or C-terminal end are identified along with the molecular weight of the unidentified protein may be compared to the protein population to identify the unknown protein. Alternatively, embodiments in which the first 10 or fewer amino acids from the N-terminal or C-terminal end are identified along with the molecular weight of the unidentified protein are compared to the protein population to identify the unknown protein. Alternatively, embodiments in which the first 20 or fewer amino acids from the N-terminal or C-terminal end are identified along with the molecular weight of the unidentified protein are compared to the protein population to identify the unknown protein. Alternatively, embodiments in which the first 30 or fewer amino acids from the N-terminal or C-terminal end are identified along with the molecular weight of the unidentified protein are compared to the protein population to identify the unknown protein.

In an alternative embodiment, a protein population or protein sequence library may not be created prior to the sequencing of unknown samples. For example, one or more samples may be processed in a manner that identifies the first 5 or fewer amino acids in sequence, the first 6 or fewer amino acids in sequence, the first 10 or fewer amino acids in sequence, or the first 20 or fewer amino acids in sequence, along with the molecular weight of the one or more unknown protein samples. Once the initial amino acid sequence is identified, a mapped genome may be analyzed to identify all potential proteins that may be produced by the organism in question, or an RNA, DNA, or known protein samples may be probed to identify a protein population that has an identical initial sequence by, for example, a BLAST search.

According to yet another embodiment of the present application, a short series of ladder sequencing may be utilized to cleave an unknown sample that has been bonded or attached to a solid surface (such as a membrane) into several different sized polypeptide fragments. Once the free fragments are washed, the solid surface may be subjected to mass spectrometry to identify the sequence of a certain number of amino acids within the protein. The location of these identified amino acids, along with the molecular weight of the sample, can then be compared against a previously generated protein sequence library as discussed above, or may be used to probe a genome, RNA, or DNA as previously discussed to identify an unknown protein sample.

It will be appreciated that each of the above embodiments can be performed by obtaining a relatively pure protein sample from a mixed protein sample by utilizing a 2D separation, such as gel electrophoresis or chromatography, to separate out the various proteins in an unknown sample into its individual protein samples. Alternatively, a 1D separation may be performed, with the mass differences of the proteins in a mixed sample may be utilized to identify the multiple proteins in a mixed sample, although such a mixture will complicate analysis of the sample.

### Example

An exemplary embodiment of one aspect of the present application would involve the use of an unknown mixed protein sample. The unknown mixed protein sample is subjected to a 2D separation, and a purified protein sample is obtained by pulling out one of the samples from the 2D separation—which should hold several molecules of a particular unknown protein. The sample is then adhered to a membrane attached to a reaction vessel and run through an automated system as described below. Reagents are selected to perform a sequencing described above to obtain the sequence of the first 6 N-terminal amino acids. After washing the reagents and optionally saving the eluted cleaved peptides and amino acids from the reactor vessel, the remaining fragments still attached to the film are subjected to mass spectrometry to determine the sequence of the first 6 N-terminal amino acids and the molecular weight of the fragments, from which the molecular weight of the entire sample can be derived, if necessary.

In this example, the amino acid sequence for the first 6 amino acids is GDPGGV. A search of a known protein database for the 6 amino acid sequence, in this instance, the database maintained for proteins at the National Center for Biotechnology Information, is searched for the GDPGGV sequence. A total of 46 possible proteins are identified when a search of this 6 amino acid sequence is performed. Additionally, the 46 possible proteins identified include proteins from several different organisms. This list can be substantially reduced by removing all but the known organism from which the sample was taken, if known. Additionally, the number in the protein sequence where the GDPGGV sequence is found is identified in the database, so it can be determined whether the unknown protein was later phosphorylated or otherwise changed from its original state in the organism from which it came.

In the event that no such results are present for a given sequence, a DNA or RNA probe corresponding to the amino acid sequence can be created to identify the sequence in the organism's DNA that codes for the protein, thereby allowing the identification of the protein.

It will be appreciated that acid and base labile xanthates provide a more robust means of limited protein and peptide amino terminal sequencing in conjunction with mass spectroscopy due to the fact that side chain amino acids are not chemically modified. Additionally, free N-terminal amino-acid without any derivitaization are obtained by using these methods, and these amino acids are available for confirmation of molecular weight ladder sequencing obtained by mass spectroscopy, and these amino acids also provide a means of differentiating between ambiguous sequence assignments in mass spectroscopy such as between leucine and isoleucine, lysine and glutamine. Furthermore, during selective degradation of peptides or protein by these reagents, the side chain amino-acid groups of proteins and peptides are easily recovered from their derivatized state. Further, while this application describes the desirable attributes of soluble acid and base labile xanthate reagents, practitioners of the art in protein sequencing chemistry will readily appreciate that extension of this chemistry to such acid and base labile and photo labile xanthate reagents obtained or prepared as reactive solid surface reagents, will impart all the desirable characteristics of these reagents to applications in protein sequencing to such immobile solid surfaces as described herein for the soluble reagents, and therefore, application of such labile xanthate solid supports to protein sequencing are incorporated in this application by extension to those practices in the art. Finally, it will be appreciated that recovered free amino-acids from sequencing cycles can be detected with much greater sensitivity in the sub femtomole & attomole range.

What is claimed is:

1. A protein sequencing system comprising:
a. a protein sequence library from an organism or group of organisms from which a plurality of unknown protein samples are taken;
b. a protein sequencer having a plurality of reactor vessels in fluid communication with at least one fluid inlet port and wherein each reactor vessel is operable to retain one of the plurality of unknown protein samples;
c. at least one cleavage reactant comprising an acid or base labile xanthate operable to cleave one or more amino acids from the unknown protein samples and result in a remaining polypeptide or dipeptide.

2. The system of claim 1, wherein the at least one cleavage reactant, when applied to a protein or polypeptide sequence, results in a O-tertiary butyl xanthamido derivative of an α amino acid derivative of the protein or polypeptide sequence.

3. The system of claim 1, wherein the at least one cleavage reactant, when applied to a protein or polypeptide sequence, results in a O-(9-H-Fluoren-9-yl) xanthamido derivative of an α amino acid derivative of the protein or polypeptide sequence.

4. The system of claim 1, wherein the acid or base labile xanthate is selected from the group comprising methyl o-fluorenylmethyl xanthate and methyl o-t butyl xanthate.

5. The system of claim 1, wherein the cleaved one or more amino acids results in a xanthamido derivative of the α amino present in the protein or polypeptide sequence, and wherein the addition of an acid or base results in uncoupling the acid or base labile xanthate from the cleaved one or more amino acids.

6. A method for sequencing a protein or polypeptide sequence, comprising the steps of:
a. providing a polypeptide sample to be sequenced;
b. providing at least one cleavage reactant comprising an acid or base labile xanthate operable to cleave one or more α amino acids from the polypeptide and resulting in a xanthamido derivative of the one or more α amino acids;
c. separating the xanthamido derivative of the one or more α amino acids from the remaining polypeptide;
d. exposing the xanthamido derivative of the one or more α amino acids to an acid or a base, resulting in the free form of one or more α amino acids; and
e. analyzing the free form of the one or more α amino acids using mass spectroscopy.

7. The method of claim 6, wherein the acid or base labile xanthate is selected from
the group comprising methyl o-fluorenylmethyl xanthate and methyl o-t butyl xanthate.

8. The method of claim 6, wherein steps a through e are performed sequentially, and
repeated a predetermined number of times.

9. The method of claim 8, further comprising the step of obtaining a protein sequence library from an organism or group of organisms from which the polypeptide samples was taken, and comparing each of the α amino acids identified via mass spectroscopy against the protein sequence library to identify the polypeptide sample.

10. The method of claim 9, wherein the protein sequence library from an organism or group of organisms is obtained from a BLAST search.

11. The method of claim 9, further including the step of providing a protein sequencer having a plurality of reactor vessels in fluid communication with at least one fluid inlet port and wherein each reactor vessel is operable to retain one of the plurality of unknown protein samples.
